(19) United States
(12) Patent Application Publication
(10) Pub. No.: US 2016/0000512 A1
(43) Pub. Date: Jan. 7, 2016

(54) HOLDING DEVICE FOR A SURGICAL INSTRUMENT AND A SHEATH AND METHOD AND CONTROL DEVICE FOR OPERATING A ROBOT WITH SUCH A HOLDING DEVICE

(71) Applicant: GOMTEC GMBH, Seefeld (DE)

(72) Inventors: Bernd Gombert, Worthsee (DE); Patrick Rothfuss, Hallbergmoos (DE); Leopold Krausen, München (DE); Martin Schautt, München (DE)

(21) Appl. No.: 14/768,806

(22) PCT Filed: Jan. 30, 2014
(86) PCT No.: PCT/EP2014/051768

§ 371 Date: Aug. 19, 2015

(30) Foreign Application Priority Data
Feb. 19, 2013 (DE) 10 2013 002 818.3

(51) Int. Cl. A61B 19/00

(52) U.S. Cl. CPC A61B 19/2203; A61B 2017/00477

(57) ABSTRACT

The invention relates to a device (10) for holding a surgical instrument (11), which is designed to be introduced through a sheath (12) into the body of a patient (2), and a sheath (12). The holding device according to the invention comprises a first holder (26) for the surgical instrument and a second holder (18) for the sheath. Furthermore, a drive unit (27) is provided by means of which said second holder (18) can be moved relative to said first holder in the longitudinal direction (15) and/or the direction of rotation of the surgical instrument (11).

The invention relates to a device for holding a surgical instrument, and a sheath according to the preamble of patent claim 1, and a method and a control device for operating a robot with a robotic head, on which such a holding device is fixed, according to the preamble of patent claims 11 and 17.

Surgical procedures on the human body are now performed increasingly in minimally invasive procedures with the assistance of surgical robots. Depending on the type of procedure, the surgical robots can be outfitted with various surgical instruments, such as endoscopes, trocars, cutting, gripping or sewing instruments. During the operation, the instruments are inserted into the body of the patient, where the surgical procedure then takes place, by means of one or more robots via a sheath. The robotic system is thereby controlled by a surgeon or where appropriate also by a surgical team via an input device.

FIG. 1 shows a typical robotic system 1 having a surgical robot 4 which is designed for a minimally invasive procedure on a patient 2. The patient 2 lies on an operating table 3 and is treated by the surgical robot 4 which comprises a multi-member robotic arm with arm members 8, 9 which are connected to each other via a joint 5, 6. On the robotic head 7 is fixed a holding device for various surgical instruments and equipment 11. As can be seen, a surgical instrument 11 is inserted inside the body through a trocar sleeve 12 which has been placed into the body of the patient 2.

From U.S. Pat. No. 7,955,322 B2 (see FIG. 2) a robotic arm 100 is known with a holding device 101 for a surgical instrument 103, on which is also fixed a trocar sleeve 102. The holding device 101 further comprises a carriage, by means of which the surgical instrument 103 is displaceable in its axial direction (arrow 105). The instrument 103 can thus be displaced in the axial direction independently of the robotic arm. However, during movement of the robotic arm 100, both the surgical instrument 103 and the trocar sleeve are always moved simultaneously. If, for example, the position of the robotic head is displaced in the longitudinal direction of the surgical instrument 103, the trocar sleeve 102 follows along with this movement, and can thus be pulled out of or inserted too far into the body of the patient. The freedom of movement of the robot 100 during an operation is therefore limited to pivoting movements. Moreover, it is disadvantageous in this construction of the holding device 101 that the trocar sleeve 102 performs a rotational movement when the robotic head is rotated about the longitudinal axis of the instrument 103. The trocar sleeve 102 then rubs against the surrounding tissue such that this can be damaged.

From DE 196 09 034 A1 a device is known for holding a surgical instrument and a sheath. If such a holding device is fixed onto a surgical robot, the movement of the robot is transferred both to the surgical instrument as well as to the sheath. It is therefore not possible to move the surgical instrument by movement of the robot in its longitudinal direction, as otherwise the sheath would be inserted more deeply into the patient or pulled out from the body of the patient.

It is thus an object of the present invention to provide a holding device for holding a surgical instrument and a sheath in which the surgical instrument can be moved in the longitudinal direction during an operation by displacement of the robotic arm. Alternatively or additionally, the holding device according to the invention should be designed such that the sheath rubs less strongly against the surrounding tissue if the robotic arm (including the robotic head) executes a rotational movement about the longitudinal axis of the surgical instrument or a longitudinal movement.

This object is achieved according to the invention by the features specified in patent claim 1, in claim 11 and in claim 17. Further embodiments of the invention result from the dependent claims.

According to the invention is proposed a device for holding a surgical instrument and a sheath, which comprises a first holder for the surgical instrument and a second holder for the sheath.

According to the invention is further provided a drive unit, by means of which the second holder is displaceable in the longitudinal direction relative to the first holder and the robot, and/or by means of which the sheath can be rotated about its longitudinal axis. The sheath is therefore movable independent of the surgical instrument in the longitudinal direction thereof and/or rotatable about its longitudinal axis. This construction has the advantage that a longitudinal or rotational movement of the robotic arm (including the robotic head) can be compensated by a corresponding counter-movement of the sheath, so that the sheath can be held still relative to the body of the patient and thus the surrounding tissue at the incision site is not irritated or damaged.

By a “sheath” is to be understood within the context of this document in particular any device which is designed to provide entry for one or more surgical instruments into the body of a patient. According to the invention, sheaths may be trocar sleeves, for example, or any other sleeve-like elements, such as so-called ports. According to the present invention, the terms “sheath,” “trocar sleeve” and “port” can therefore be used interchangeably.

By a “robot” is to be understood within the context of this document in particular a device with one or more articulated arms, which are movable by means of one or more actuators, for example electric motors. The degree of freedom of the robot is determined by the number of its joints. The robot is advantageously designed as a so-called robotic arm, the last member of which can be designated as a robotic head.

According to a preferred embodiment of the invention, the holding device comprises at least a first drive unit by means of which the second holder is displaceable relative to the first holder in the longitudinal direction of the surgical instrument. This first drive unit preferably comprises a motor-transmission unit with an electric motor and a mechanical transmission.

Alternatively or additionally, the holding device can also comprise a second drive unit, by means of which the sheath can be rotated about its longitudinal axis or the longitudinal axis of the surgical instrument. The second drive unit also preferably comprises a motor-transmission unit with an electric motor and a mechanical transmission.

According to a specific embodiment of the invention, the second drive unit can comprise an electric motor and a sleeve rotatably driven by the electric motor, in which the sheath is arranged so as to be secure from rotation.

The first or second drive unit could alternatively comprise a hydraulically or pneumatically actuated drive apparatus.

The first drive unit for displacing the sheath in the longitudinal direction of the surgical instrument is preferably integrated in the holding device according to the invention, and in particular at least partly integrated in the first holder for the surgical instrument. However, it may also be integrated into the robot, for example.

The second drive unit for performing a rotational movement of the sheath is preferably integrated in the holding device according to the invention, and in particular at least partly integrated in the second holder. It is therefore brought along during a longitudinal movement of the sheath in the longitudinal direction of the surgical instrument. However, it may also be integrated into the robot, for example.

The second holder for the sheath preferably comprises a clamping device. The clamping device can comprise, for example, two opposite-facing clamping jaws, between which the surgical instrument can be clamped in.

The holding device according to the invention preferably also comprises a unit for fixing the holding device to a robot. Any known fastening device, such as a lock-in, plug-in, clamp-in or screw-in connection, can in principle be used for fixing the holding device. According to a specific embodiment of the invention may also be provided a quick-connect mechanism, by means of which the holding device can be mounted, in particular without a tool, on the robotic head. The quick-connect mechanism can comprise, for example, a known tongue over-center nut mechanism or another clamping mechanism, as is known in various embodiments from the prior art.

The holder for the surgical instrument is preferably permanently integrated in the holding device, and thus is arranged, for example, fixedly in relation to the fixing device. According to a specific embodiment of the invention, the first holder can also be arranged movably in the longitudinal direction and/or direction of rotation. In this case, a corresponding additional drive unit is preferably provided.

The holding device according to the invention preferably also comprises an electrical and/or mechanical interface, via which forces, torques, electrical variables and/or data can be transferred from or to the surgical instrument or from the surgical instrument to a control unit of the robot.

According to an embodiment of the invention, the holding device comprises a guide which is integrated fixedly (immovably) in the holding device. The second holder for the sheath is slidably arranged on the guide such that it can move in the longitudinal direction of the surgical instrument. The fixed guide extends in the longitudinal direction of the instrument preferably at least over a distance corresponding to the displacement area of the second holder in the longitudinal direction.

According to another embodiment of the invention, the second holder comprises a cantilevered arm that is movably arranged relative to the first holder. A holding unit, such as a clamp, to which the sheath is fixed, is preferably provided on this arm. In this embodiment, the entire arm including the holding unit and the sheath are driven in the longitudinal direction of the surgical instrument from the first drive apparatus. The cantilevered arm is preferably mounted displaceably in the longitudinal direction in the region of the first holder. The arm is preferably also designed such that it does not protrude at the distal end of the holding device over the holding unit for holding the sheath. As a result, the sheath can be more deeply inserted into the patient.

In principle, the holding device according to the invention offers the possibility of compensating a movement of the robotic head in the longitudinal direction of the surgical instrument and/or a rotational or pivoting movement of the robotic head about the point of incision of the sheath, in that the sheath performs a corresponding counter-movement. The proposed holding device also offers the possibility for the compensation of further forms of movement which can cause a relative movement of the sheath at the point of incision, for example, a longitudinal or rotational movement of the surgical instrument itself, or a corresponding movement of the body of the patient. According to the invention is therefore proposed a method for operating a robot, to which a holding device as described above is fixed, with which relative movements of the sheath relative to the patient can be avoided as much as possible.

According to the invention is proposed a method for operating a robot, in which the robotic head is displaced by corresponding control of the robot in the longitudinal direction of the surgical instrument and the holder for the sheath is displaced by corresponding control of the related drive unit in counter-movement to the movement of the robotic head, such that the two movements are substantially, preferably completely, compensated.

The robotic system is preferably designed such that a control instruction entered by the operator of the robotic system will be implemented both in a first control command for controlling the robot and/or the robotic head as well as in a second control command for controlling the sheath. A controller thus processes the control instruction executed by the operator, and produces both a control command for controlling the robot as well as a second control command for controlling the sheath drive. The robot and the sheath are thus driven based on the same control command.

Alternatively, the movement of the robot and the robotic head can be detected by sensors, for example by means of optical sensors, such as a camera, or by means of displacement, angle or other motion sensors. In this case, a corresponding sensor would be provided that detects a movement of the robot or the robotic head (or an element fixed on the robot) and generates corresponding movement data which are taken into account by a controller for the sheath drive so as to compensate the movement of the robot.

Alternatively or in addition, a method is proposed in which the robotic head is rotationally driven about the point of incision by corresponding control of the robot and in which the sheath is rotated in the opposite direction by corresponding control of the second control unit, so that the sheath remains substantially still relative to the surrounding tissue, i.e. no relative movement of the sheath with respect to the surrounding tissue occurs. The tissue at the point of incision is thus less heavily strained.

In a specific embodiment of the holding device, the surgical instrument itself (without changing the position of the robotic head) can also be rotationally driven by means of an associated drive. In order to avoid entrainment of the sheath by the rotational movement of the surgical instrument, the drive unit of the sheath, and thus the sheath itself, can be fixed such that there is again no relative movement of the sheath with respect to the surrounding tissue.

The entire holding device is preferably pivotally attached to a robot. In this case, the first holder for the surgical instrument is preferably arranged such that the pivot axis of the holding device intersects the longitudinal axis of the surgical instrument, preferably perpendicularly. As a result, the end effector moves along a circular path about the pivot axis as a center.

If the surgical robotic system also offers the possibility to move the body of the patient—for instance through control of an adjustable operating table—it is proposed to also compensate such a movement by corresponding control of the first and/or second drive unit of the holding device. For this purpose the control instruction of the operator can in turn be implemented as well as both a first control command for controlling the device for moving the patient and a second control command for controlling the sheath. Alternatively, however, a movement of the body could also be detected by sensors, for example, and the first and/or second drive unit for driving the sheath could be correspondingly controlled.

The invention further relates to a control apparatus for controlling a robot for minimally invasive surgery on which a holding device according to the invention is fixed, having a first control unit for controlling the robot and a second control unit for controlling the first and/or second drive unit of the sheath.

### BRIEF DESCRIPTION OF THE DRAWINGS

The invention is further explained below by way of example with reference to the accompanying drawings.

FIG. 1 shows an operating room with a known robot for minimally invasive surgery;

FIG. 2 shows a robot known from the prior art with a holding device which has a carriage for displacing the position of the surgical instrument and a holder for a trocar sleeve;

FIG. 3 shows a robotic head with a holding device fixed thereon for a surgical instrument and a sheath according to a first embodiment of the invention;

FIG. 4 shows a longitudinal section through the arrangement of FIG. 3;

FIG. 5 shows the arrangement of FIG. 3 in a state in which the sheath is located at a distal end of the holding device;

FIG. 6 shows the arrangement of FIG. 3 in a state in which the robotic head rotates about the point of incision with respect to the longitudinal axis of the surgical instrument;

FIG. 7 shows the operating room of FIG. 1, after the body of the patient has been moved into another position;

FIG. 8 shows a view of the holding device for a surgical instrument according to the invention and a sheath according to a second embodiment of the invention, wherein the sheath is located in a forward (distal) position;

FIG. 9 shows the arrangement of FIG. 8, in which the sheath is located in a middle position; and

FIG. 10 shows a sectional view of the arrangement of FIG. 9.

### EMBODIMENTS OF THE INVENTION

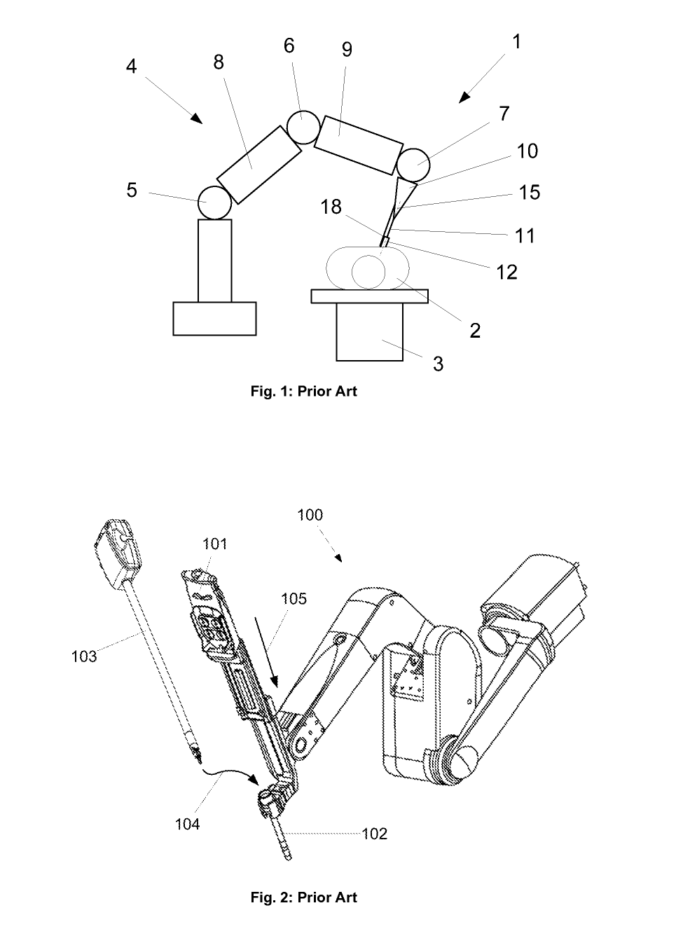

With regard to the explanations of the FIGS. 1 and 2, reference is made to the introductory part of the specification.

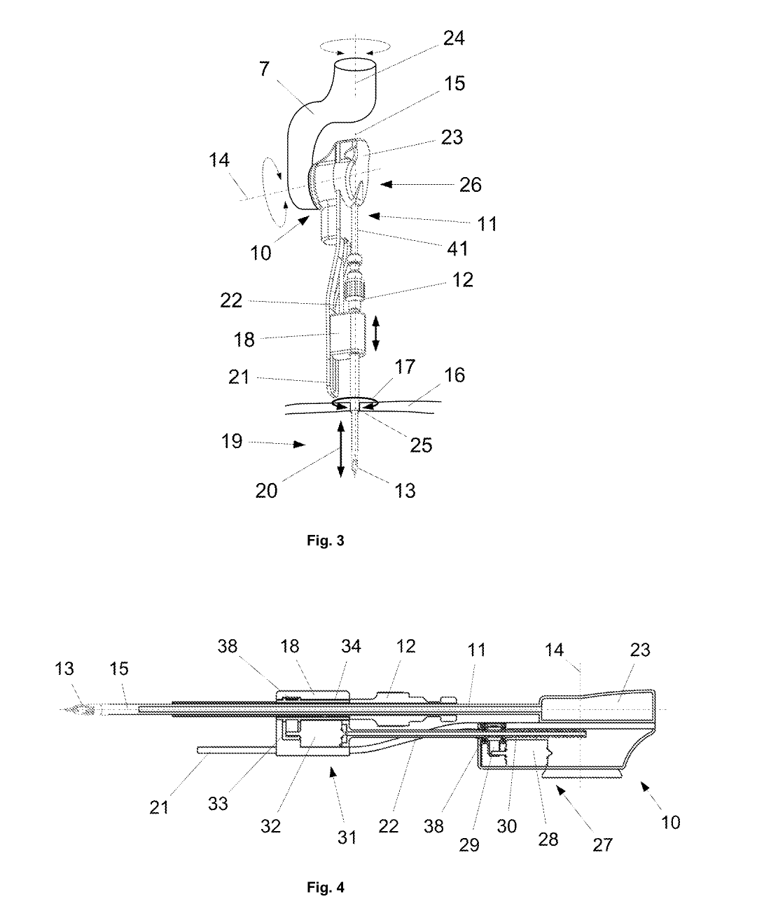

FIG. 3 shows an enlarged view of a robotic head 7 with a releasable holding device 10 fixed thereon for holding a surgical instrument 11 and a sheath or trocar sleeve 12.

The holding device 10 comprises a first holder 26 for a surgical instrument 11 and a second holder 18 for a sheath 12. Depending on the type of the instrument 11, it may comprise a different end effector 13 and be configured, for example, as a gripping, holding, cutting, sawing, grinding, connecting, disposing, optical or other tool, such as a scalpel, an angle cutter, tongs, a trocar, etc. The sheath in this case is a trocar sleeve, but can also be another device, such as a port, which is designed to receive the shaft 41 of the surgical instrument 11 and to lead it in the longitudinal direction 15 thereof.

In the illustrated exemplary embodiment, the holder 26 for the surgical instrument 11 is located at a proximal end (above in this case) of the holding device 10 and comprises a receptacle into which the surgical instrument 11 can be inserted. The instrument 11 is at least partially protected towards the outside by a housing 23 with a cover.

In an inserted state, the surgical instrument 11 is fixed in the longitudinal direction with respect to the holding device 10. The instrument 11 can, however, optionally rotate the shaft 41 and the end effector 13 connected therewith about its longitudinal axis 15. The shaft 41 of the surgical instrument 11 is inserted through the trocar sleeve 12 and protrudes at the distal end of the trocar sleeve 12 (pictured below) into the body interior 19 of a patient 2. The trocar sleeve 12 is thereby inserted, for instance, into the point of incision 25 (also called a trocar point) of the abdomen 16 of the patient 2. The actual end effector, such as a gripper, is indicated by the reference character 13.

The first holder 26 preferably comprises an interface by means of which all required physical quantities such as forces, torques, currents, or information can be transmitted, in order to actuate the surgical instrument 11 including the end effector 13.

The second holder 18 is formed here as a sleeve-like mounting into which the trocar sleeve 12 can be inserted.

The robot 4, on which the holding device 10 is fixed, preferably has so many degrees of freedom as to be able to move the holding device 10 freely. The free end of the robot 4, also called a robotic head 7, can rotate here by means of example about an axis 24. The holding device 10 can additionally be pivoted about an axis 14 of the robotic head 7.

If the robotic head 7 is moved for example upward or downward in the longitudinal direction 15 of the surgical instrument 11, the entire holding device 10 including the surgical instrument 11, the second holder 18 and the trocar sleeve 12 are taken along. The axial movement of the robotic head 7 is indicated by an arrow 20. If, on the other hand, the robot 4 or the robotic head 7 is pivoted about the axis 24 or about the point of incision 25, the entire holding device 10 is likewise taken along. The rotational movement of the robotic head 7 is indicated by an arrow 17.

Each movement of the holder device 10 in both the longitudinal direction 15 and in the direction of rotation 17 has the result that the trocar sleeve 12 moves at the point of incision 25 relative to the surrounding tissue. The patient may thereby be hurt or the tissue may at least be damaged. In order to avoid a relative movement of the trocar sleeve 12 with respect to the surrounding tissue, the holding device 10 according to the invention comprises at least one drive unit. The holding device 10 illustrated herein comprises two drive units 27 and 31 (see FIG. 4), with which an axial movement 20 and/or rotational movement 17 of the robotic head 7 can be compensated. In the best case, the trocar sleeve 12 can thus be held still relative to the patient.

To compensate for axial movements 20 of the robotic head 7, the holder 18 is arranged on a fixed guide 21. The guide 21 is formed here in a rail-like manner and immovably fixed to the holding device 10. It extends in the longitudinal direction 15 of the instrument 11 at least over a distance corresponding to the displacement area of the second holder 18. The second holder 18 is slidably arranged on the guide 21 and is displaceable in a longitudinal direction 15 relative to the first holder 10 by means of the first drive unit 27. By means of corresponding control of the first drive unit 27, an axial movement 20 transferred from the robotic head 7 to the trocar sleeve 12 can thus be partly or completely compensated at the point of incision 25. In an ideal case, the trocar sleeve 12 then remains fixed in the longitudinal direction 15, so that no relative movement occurs between the trocar sleeve 12 and the surrounding tissue.

To compensate for rotational movements of the robotic head 7, a second drive unit 31 is provided, by means of which the trocar sleeve 12 can be rotated about its longitudinal axis. Rotational movements of the robotic head 7 can thus also be at least partially or completely compensated. In the best case, no relative movement in turn occurs between the trocar sleeve 12 and the surrounding tissue. The operation can therefore be very gently carried out.

In FIG. 4, the two drive units 27 and 31 are shown in greater detail. In the exemplary embodiment shown, the drive unit 27 for compensating an axial movement 20 comprises a motor-transmission unit with an electric motor 28, which drives a spindle 30 via a transmission 29, whose longitudinal axis runs substantially parallel to the longitudinal axis 15 of the surgical instrument. The spindle rod 30 is partially formed as a push rod 22 which is mechanically connected to the holder 18. However, the spindle 30 and push rod 22 could also be designed as separate parts. By moving the spindle rod 30 forward or backward in the longitudinal direction, the holder 18 moves along the guide 21 in the axial direction 15 of the surgical instrument 11. The first drive unit 27 is integrated here in a common housing with the first holder 26.

Instead of the spindle drive, an alternative drive could also be selected, for instance a rack and pinion drive or toothed belt drive or a hydraulic or pneumatic drive.

The second drive unit 31 is preferably also designed as a motor-transmission unit and comprises an electric motor 32 which drives a sleeve 34 in the direction of rotation 17 via a transmission 33. The sleeve 34 is designed such that the trocar sleeve 12 can be inserted herein in a manner secure from rotation.

In the illustrated exemplary embodiment, the second drive unit 31 is integrated in the holder 18 for the trocar sleeve 12. By means of the second drive unit 31, the sleeve 34 and thus also the trocar sleeve 12 can thus be rotated about the longitudinal axis 15.

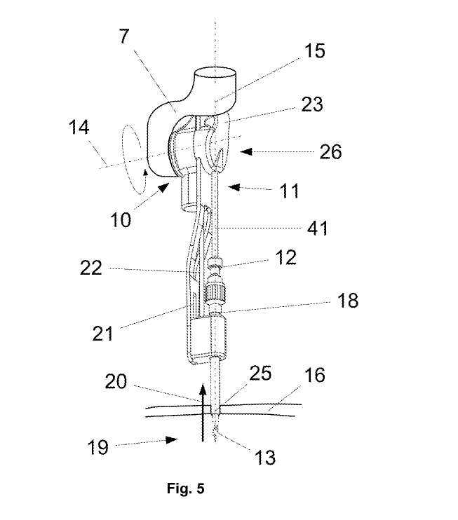

FIG. 5 shows the robotic head 7 and the holding device 10 in a state in which the holding device 10 is somewhat more raised as compared to FIG. 3 and the second holder 18—in order to compensate for this movement—has been moved further downward along the guide 21. The oppositely directed movements of the robotic head 7 and the holder 18 preferably occur synchronously and to the same extent, so that the trocar sleeve 12 received in the holder 18 does not undergo any relative movement at the point of incision 25. No (frictional) forces thus act upon the patient 2 through the trocar sleeve 12.

If, however, the surgical instrument 11 is inserted further into the patient 2 through a corresponding lowering of the robotic head 7, the holder 18 is moved further upward by means of the first drive unit 27, as shown in FIG. 3. In turn, the trocar sleeve 12 remains fixed in place with respect to the patient 2.

During a rotation of the robotic head 7 about the axis 24 (which in the illustrated state coincidentally aligns with the longitudinal axis 15 of the surgical instrument 11), the trocar sleeve 12 is automatically rotated in the opposite direction. If, for example, the holding device 10 is rotated or pivoted to the right by a rotary movement of the robotic head 7, the trocar sleeve 12 according to the invention is rotated by a corresponding angle to the left by means of the drive unit 31, so that the rotational or pivoting movements transferred from the robotic head 7 to the trocar sleeve 12 are compensated at the point of incision 25. The same applies if the robotic head 7 is rotated or pivoted to the left. Through the opposing movement of the trocar sleeve 12, frictional torque can thus in turn be prevented at the point of incision 25.

Because the trocar sleeve 12 is generally in direct contact with the surgical instrument 11, there also exists the possibility that, as a result of a compensating movement, the trocar sleeve 12 as well as the instrument 11 and the shaft 41 thereof are moved as well. A rotational movement of the trocar sleeve 12 would in this case unintentionally displace the position of the end effector 13. It is therefore proposed, during a movement of the trocar sleeve 12, that the instrument 11 also be driven in such a way that the surgical instrument 11 and the end effector 13 maintain their position with respect to the holding device 10. To this end, the surgical instrument 11 can, for example, move the shaft 41 contrary to the trocar sleeve 12, or a force or a torque can be exerted which compensates the force or the corresponding torque exerted by the trocar sleeve 12.

To exercise the appropriate counter-force or counter-torque, one or more sensors may be provided that detect, for example, a change in the position of the instrument 11 or the end effector 13. The counter-force or counter-torque could then be increased or decreased depending on the measured movement.

Through a displacement of the trocar sleeve 12, it is not only possible to compensate movements of the robotic head 7 or the holding device 10, but also movements of the surgical instrument itself. Instruments 11 are known from the prior art which can actuate the end effector 13 both axially and rotationally. The forces or torques required for this purpose are typically transferred to the end effector 13 via the instrument shaft 41.

In order to now counter an unintentional movement of the trocar sleeve 12 caused by the longitudinal or rotational movement of the surgical instrument 11, the trocar sleeve 12 can be held by means of the first or second drive unit. This means that the drive units generate forces or torques which oppose the forces or torques transferred from the instrument 11 to the trocar sleeve 12. The trocar sleeve 12 can thus be held unchanged in position while the instrument 11 is moved. This is easily implemented by means of a conventional control.

Through the design of the holding device 10 according to the invention, it is also possible to compensate movements of the patient 2 relative to the holding device 10. FIG. 7 shows an operating room with a robotic system 1 for minimally invasive surgery in which a patient 2 lies on an electrically adjustable operating table 3 and is treated by a robot 4 which is equipped with a surgical instrument 11. The body of the patient 2 has been rotated in its position in comparison to FIG. 1 by means of the operating table 3. In order to minimize the occurrence of friction of the trocar sleeve 12 against the surrounding tissue at the point of incision 25 during such a movement of the patient 2, the trocar sleeve 12 is preferably rotated counter to the rotational movement of the patient. A change in position of the patient 2 can thus be compensated by corresponding counter-movement of the trocar sleeve 12. The same applies to a movement of the patient in the longitudinal direction 15 of the surgical instrument 11.

In order to displace the trocar sleeve 12 in the desired manner, for example, the control data may be used with which the operating table 3 or other device for moving the body of the patient 2 is displaced. From these data, the corresponding control commands for controlling the trocar sleeve 12 can then be generated.

Alternatively, the movement of the body of the patient 2 may also be detected sensorially, for instance by means of a camera or other sensor. From the sensor information the change in position of the patient 2 and appropriate control commands generated therefrom can then in turn be determined for a movement of the trocar sleeve 2.

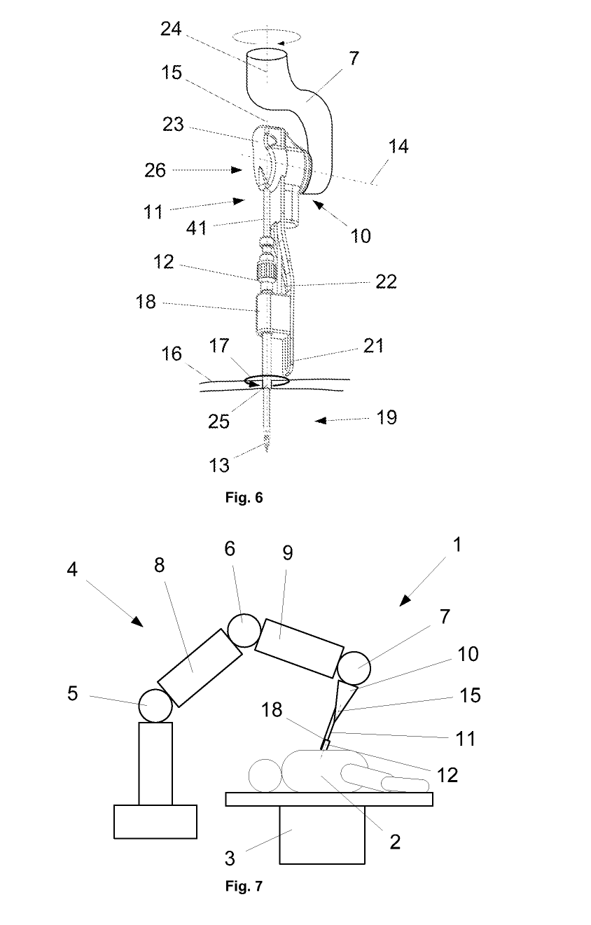

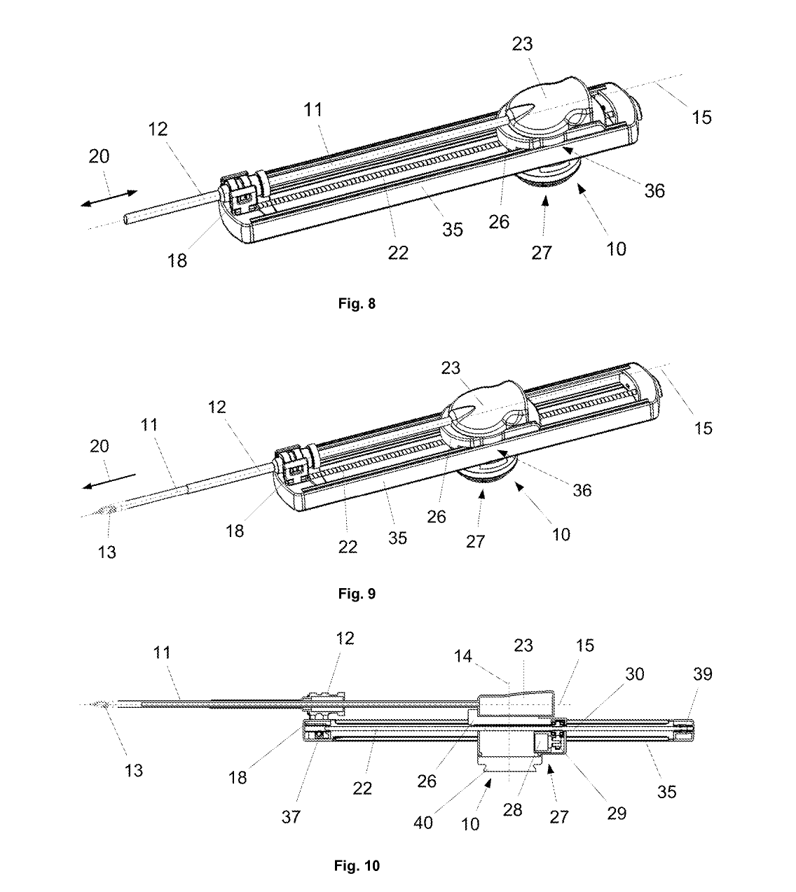

FIG. 8 shows a second embodiment of a holding device 10 for a surgical instrument 11 and a sheath 12. As in the embodiments of FIGS. 3 to 6, the second holder 18 is displaceable in the longitudinal direction 15 of the surgical instrument 11 relative to the first holder 26 for the surgical instrument 11. In contrast to the embodiment of FIGS. 3 to 6, the holder 18 comprises a cantilevered arm 35, which extends approximately over a distance which corresponds to the displacement area of the second holder in the longitudinal direction. The holding unit 18 is fixed to the arm 35. The arm 35 is guided in the area of the first holder 26 and is axially movably mounted there by means of a bearing 36.

Analogous to the first embodiment a drive unit 27 is provided again for axial displacement of the second holder 18, 35 and thus the trocar sleeve 12. The drive unit 27 comprises a motor-transmission unit which comprises a push rod 22 which is mechanically coupled to the arm 35, so that the entire arm 35 and the sheath 12 fixed thereon by means of the holder 18 can be displaced along the longitudinal axis 15.

The arm 35 is preferably designed such that it does not protrude (at the distal end of the holding device 10) over the actual holding unit 18 for holding the sheath 12. As a result, the sheath 12 can be more deeply inserted into the patient 2 in comparison with the first exemplary embodiment.

In FIG. 8, the arm 35 is in a position moved far toward the patient 2. FIG. 9 shows the holding device 10 of FIG. 8 in a middle position. In comparison with FIG. 8, the sheath 12 has been moved in the direction of the first holder 26. As a result, the surgical instrument 11 has moved further forward with respect to the sheath 12, as indicated by an arrow 20.

Finally, FIG. 10 shows a sectional view of the arrangement of FIG. 9, in which the individual components of the drive unit 27 for displacing the trocar sleeve 12 in the axial direction are particularly clearly visible. The drive unit 27 comprises here an electric motor 28 which drives a spindle rod 30 via a transmission 29. The spindle rod 30 is supported in a rotation-proof manner in the arm 35 at both its distal and at its proximal end. The two bearings are designated by reference numerals 37 and 39, respectively.

The embodiment illustrated in FIGS. 8 to 10 can also be extended by a drive unit 31 in order to exert a rotational movement on the sheath 12 in accordance with the first embodiment and thus to avoid friction between the sheath 12 and the surrounding tissue.

Each holding device 10 according to the invention may in principle be equipped with one or more sensors, with which position, motion, acceleration, force and/or torque can be determined. Such a sensor may, for example, be integrated in the drive unit 27 and/or 31. In the case of a position sensor, for example, the position of the sheath 12 or the holder 18 with respect to the holder 10 can be determined. The sensor information can be monitored and a safety function can be executed, for example, when a critical value has been determined. For instance, in the case of excessive force upon the holding device 10, an automatic emergency stop can be initiated. The sensor data can further be used to check whether the sheath 12 has also been displaced in accordance with the associated control command. If, for example, the actual position or movement of the sheath differs from the desired position or movement, the deviation can be regulated, e.g. by means of a closed-loop control. The same applies in the event of force or torque sensing.

Finally, FIG. 10 additionally shows a fixing device 40 for fixing the holding device 10 on a robot. The fixing device 40 can be a part of a screw connection, comprising for example a screw, a screw hole or a threaded hole, etc. The fixing device 40 are, however, preferably designed as a quick-connect mechanism, such as a tongue-and-groove connection with a movably arranged tongue, or a known an over-center connection with a tongue and an over-center nut, a locking connection or a known quick-release connection, for instance with a clamping lever. The connection mechanism can preferably be operated without tools.

1. A device for holding a surgical instrument having a shaft which extends in a longitudinal direction and a sheath, comprising a first holder for the surgical instrument and a second holder for the sheath, and a first drive unit, by which the second holder is displaceable in the longitudinal direction of the surgical instrument, relative to the first holder, wherein a guide is provided which is fixed in relation to the first holder and which connects the first holder and the second holder mechanically with one another, and wherein the second holder can slide on the guide such that it is displaceable in the longitudinal direction of the surgical instrument.

2. The device according to claim 1, wherein the first drive unit is designed as a motor-transmission unit comprising an electric motor and a mechanical transmission.

3. The device according to claim 1, wherein the first drive unit is integrated in the first holder.

4. The device according to claim 1, with a second drive unit which is integrated in the second holder and drives the sheath in rotation about its longitudinal axis.

5. The device according to claim 1, wherein the first drive unit comprises a nut and lead-screw drive.

6. The device according to claim 4, wherein the second drive unit is designed as a motor-transmission unit having an electric motor and a holding unit, in particular a sleeve, rotatably driven by the electric motor.

7. The device according to claim 1, wherein it includes a device for fixing to a robot.

8. The device according to claim 7, wherein the instrument is moved according to the motion of the robot and the sheath is moved relative to the robot.

9. The device according to claim 1, wherein it comprises an electrical and/or mechanical interface, via which forces, torques, electrical power and/or data may be transmitted.

10. (canceled)

11. A device for holding a surgical instrument having a shaft which extends in a longitudinal direction and a sheath, comprising a first holder for the surgical instrument, a second holder for the sheath and a first drive unit, by which the second holder is displaceable in the longitudinal direction of the surgical instrument relative to the first holder, wherein an arm is provided which mechanically connects the first holder and the second holder with one another, the second holder is fixed on the arm, and wherein the arm is movably arranged relative to the first holder such that the arm together with the second holder is displaceable in the longitudinal direction of the surgical instrument relative to the first holder.

12. A method for operating a robot having a robotic head to which a holding device for holding at least one surgical instrument or tool and a sheath is attached, wherein the surgical instrument has a shaft extending in a longitudinal direction, with the following steps:
Displacement of the robotic head and/or a patient treated by the robot and/or the instruments guided by the robot and;
Simultaneous execution of a counter-movement by the sheath, wherein the sheath is displaced in the longitudinal direction of the surgical instrument and/or rotated about its longitudinal axis so that the relative motion of the sheath at the point of incision of the patient caused by motion of the robotic head and/or of the patient and/or of the instrument can be compensated as much as possible.

13. The method according to claim 12, with the following steps:
Displacement of the robotic head in the longitudinal direction of the surgical instrument, and/or
Rotation of the robotic head about the longitudinal axis of the surgical instrument, and/or
Pivoting of the robotic head about the point of incision and/or
Rotation of the surgical instrument about the longitudinal axis of the surgical instrument.

14. The method according to claim 12, wherein the sheath is displaced in the opposite direction of the movement of the robotic head by means of a drive unit.

15. The method according to claim 14, wherein the sheath is displaced in the longitudinal direction of the surgical instrument by means of a first drive unit and/or is rotated about the longitudinal axis of the surgical instrument by means of a second drive unit.

16. The method according to claim 12, with the following steps:
Altering of the position of the patient in the longitudinal direction of the surgical instrument and/or
Rotation of the body of the patient about the longitudinal axis of the surgical instrument, and
Adjusting of the sheath such that the relative movement of the patient to the sheath is compensated at the point of incision of the patient.

17. A control unit comprising a device for performing the methods according to claim 13.

18. The device according to claim 10, wherein the first drive unit is integrated in the first holder.

19. The device according to claim 10, with a device for fixing on a robot, wherein the instrument is moved according to the motion of the robot and the sheath is moved relative to the robot.
